SEQUENCE LISTING (1 sequence)

SEQ ID NO: 1 - RNA, 1502 nt, Bacillus velezensis
  source 1..1502: mol_type = rRNA
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaagg

(19) United States
(12) Patent Application Publication
(10) Pub. No.: US 2026/0015635 A1
(43) Pub. Date: Jan. 15, 2026

(54) MASS PRODUCTION METHOD USING BACILLUS VELEZENSIS FOR DECOMPOSITION OF FISH SCALE AND PRODUCT THEREOF

(71) Applicant: CH BIOTECH R&D CO., LTD., NANTOU COUNTY (TW)

(72) Inventors: HUI-LIANG WANG, NANTOU COUNTY (TW); TING-WEN CHENG, NANTOU COUNTY (TW); CHENG-HUI TSAI, NANTOU COUNTY (TW); YA-FANG HONG, NANTOU COUNTY (TW)

(21) Appl. No.: 18/772,775

(22) Filed: Jul. 15, 2024

(51) Int. Cl. C12P 13/04; C12N 1/20; C12P 21/00; C12R 1/07

(52) U.S. Cl. CPC C12P 13/04; C12N 1/205; C12P 21/00; C12R 2001/07

(57) ABSTRACT

A mass production method using Bacillus velezensis for decomposition of fish scales and product of the same is provided. The Bacillus velezensis is deposited in Leibniz Institute DSMZ-German Collection of Microorganisms and Cell Cultures GmbH with an accession number of DSM 34894. The mass production method includes the steps of culturing a bacterial solution of Bacillus velezensis, mixing the bacterial solution with a fish scale material to get a mixed solution, and allowing the mixed solution to react at 37° C. for 12-24 hours for decomposition of the fish scale material. The present mass production method not only can be used to decompose a larger quantity of fish scales but also applied to mass production of a fish scale decomposition product. Thereby the present method has industrial application value.

### BACKGROUND OF THE INVENTION

### Sequence Listing Incorporated by Reference

The Sequence Listing written in file “957-3531-SEQUENCE-LISTING” created on Jul. 15, 2024, file size 3,279 bytes, is hereby incorporated by reference in its entirety for all purposes.

### Field of the Invention

The present invention relates to a mass production method and a product of the same, especially to a mass production method using Bacillus velezensis for decomposition of fish scales and a product thereof.

### DESCRIPTION OF RELATED ART

A fish scale constitutes a diminutive rigid plate emerging from a fish's skin, traditionally regarded as kitchen waste. A fish scale composition comprises 41-45% of organic substances and 38-46% of inorganic substances. The organic substances include an abundance of collagen, while the inorganic substances mainly include hydroxyapatite and calcium phosphate. Previous research has focused on the decomposition of fish scales to obtain products such as collagen, hydroxyapatite, and calcium phosphate. In most studies, the primary objective of fish scale decomposition is collagen acquisition.

A presently available method for decomposing fish scales involves enzymatic hydrolysis subsequent to acid treatment to yield collagen. Since collagen is with high molecular weight and unable to be used directly by organisms, the collagen needs to be decomposed into polypeptides for convenience of use. Previous studies have explored employing microorganisms for fish scale decomposition to directly obtain polypeptides. For example, the collagenase of Bacillus cereus is used to decompose collagen or gelatin in fish scales, fish skins, and even fish bones to obtain polypeptides.

To date, only a limited array of microorganisms is capable of fish scale decomposition, affording users few alternatives. Thus, there is room for improvement, and a necessity to identify a novel microorganism proficient in fish scale decomposition.

### SUMMARY OF THE INVENTION

Therefore, it is a primary objective of the present invention to provide a mass production method using Bacillus velezensis for decomposition of fish scales and a product of the same. The Bacillus velezensis is deposited in Leibniz Institute DSMZ-German Collection of Microorganisms and Cell Cultures GmbH with an accession number of DSM 34894. The mass production method includes the steps of culturing a bacterial solution of Bacillus velezensis, mixing the bacterial solution with a fish scale material to get a mixed solution, and allowing the mixed solution to react at 37° C. for 12-24 hours for decomposition of the fish scale material.

In a preferred embodiment, the mixed solution contains 7-10% (w/v) of the bacterial solution of Bacillus velezensis and 15-40% (w/v) of the fish scale material.

In a preferred embodiment, time for allowing the mixed solution to react at 37° C. is 16-20 hours.

In a preferred embodiment, the fish scale material includes fish scales, sodium chloride (NaCl), dipotassium phosphate (K₂HPO₄), and monopotassium phosphate (KH₂PO₄).

In a preferred embodiment, the fish scale material includes fish scales, 0.5 g/L of sodium chloride (NaCl), 0.3 g/L of dipotassium phosphate (K₂HPO₄), and 0.4 g/L of monopotassium phosphate (KH₂PO₄).

The Bacillus velezensis newly isolated by the method of the present invention possesses significant capability for decomposing fish scales, enabling its application in large-scale fermentation to effectively decompose fish scales within a short timeframe. This process results in the production of products containing high concentrations of peptides and amino acids. Moreover, the present mass production method described herein efficiently decomposes substantial quantities of fish scales, underscoring its industrial applicability and value.

### BRIEF DESCRIPTION OF THE DRAWINGS

The structure and the technical means adopted by the present invention to achieve the above, and other objects can be best understood by referring to the following detailed description of the preferred embodiments and the accompanying drawings, wherein: FIG. 1 are photos showing observation of a colony of strain CHB200, and observation of the strain CHB200 with a microscope according to the present invention;

FIG. 2 is a figure showing analysis of contents of amino acids and peptides in decomposition products of fish scales by strain CHB200 according to the present invention;

FIG. 3 are photos showing products obtained at different stages of large-scale fermentation of fish scales by strain CHB200 according to the present invention.

### DETAILED DESCRIPTION OF THE PREFERRED EMBODIMENT

In order to learn functions and features of the present invention more completely and clearly, please refer to the following detailed descriptions and related figures.

In a mass production method using a Bacillus velezensis for decomposition of fish scales according to the present invention, the Bacillus velezensis is deposited in Leibniz Institute DSMZ-German Collection of Microorganisms and Cell Cultures GmbH with an accession number of DSM 34894. 16S rRNA of the present Bacillus velezensis strain has 98% similarity with 16S rRNA of type strain (standard strain) of Bacillus velezensis B268 with an accession number of CP053764.1. This standard strain is not disclosed to have fish scale decomposing ability.

Moreover, applications of the present invention can be illustrated by the following embodiments, but not intended to limited the scope of the present invention.

### Embodiment One: Isolation and Screening of Bacillus velezensis CHB200

Collect waste chicken feathers. After washing, place 5 g of feathers into a conical flask, and add 50 mL of sterile water to get a culture medium. Place the culture medium in a 37° C. incubator, and culture overnight with a shaking speed at 200 rpm. Then, remove the feathers from the culture medium, and centrifuge the culture medium. Remove supernatant, and retain precipitate after centrifugation. Next, dissolve the precipitate with 100 μL of sterile water, and take and spread 20 μL of re-dissolved solution evenly on Lysogeny broth (LB) solid medium. Incubate the LB solid medium in the 37° C. incubator for 24 hours.

Pick a single colony grown on the LB solid medium, inoculate the colony in 3 ml of LB broth, and culture at 37° C. with a shaking speed at 200 rpm for 24 hours to get a bacterial solution.

Measure absorbance of the bacterial solution at a wavelength of 600 nm (known as OD (optical density) 600). Inoculate the bacterial solution with OD₆₀₀ of 10 therein into 100 mL of M3 fermentation medium containing 33% of fish scale powder, and ferment at 37° C. for 48 hours. Then, perform hydrolysis test of fish scales. A concentration of the above bacterial solution with OD₆₀₀ of 10 is about 1×10⁹ CFU/mL, and a composition of the M3 fermentation medium is shown in Table 1. Lastly, measure total concentration of amino acids in fermentation broth to screen out a strain with fish scale decomposition ability.

TABLE 1

| | Composition | Concentration (g/L) |
|---|---|---|
| Substrate | Fish scale | 330 |
| Salts and buffer | NaCl | 0.5 |
| | K₂HPO₄ | 0.3 |
| | KH₂PO₄ | 0.4 |

A method of testing total concentration of amino acids is described briefly below.

Dissolve leucine in double-distilled water, and prepare amino acid standard solutions with concentrations ranging from 100 ppm to 2000 ppm by serial dilution. Take 3 μL of amino acid standard solutions or test samples (fermentation broth), add 87 μL of ninhydrin reagent, and mix evenly to get an intermediate mixture. The ninhydrin reagent includes 0.6% of ninhydrin.

The intermediate mixture is reacted at 100° C. for 10 minutes. After cooling down to room temperature naturally, add 150 μL of 95% alcohol, and mix evenly to get a final mixed solution. Measure absorbance of the final mixed solution at the wavelength of 570 nm (OD₅₇₀). Lastly, make a standard curve by absorbance of a set of the amino acid standard solutions obtained. Then, total concentration of amino acids in the test sample is calculated according to the standard curve.

Among the strains tested, a strain capable of decomposing fish scales is obtained, and named as strain CHB200.

Referring to FIG. 1, a colony of the strain CHB200 is irregular round, and transparent to white colored. While being observed with a microscope, the strain CHB200 is a rod with two blunt round ends. Compare the 16S rRNA sequence of the strain CHB200 with the sequence in Genbank database, and the result shows that the strain CHB200 and the Bacillus velezensis B268 have 98% similarity in the 16S rRNA sequence. 16S rRNA of the strain CHB200 is disclosed in SEQ ID NO:1 of the sequence listing.

### Embodiment 2: Test of Fish Scale Fermentation Ability of the Strain CHB200

Inoculate the strain CHB200 into 3 ml of LB broth, and culture at 37° C. with shaking at 200 rpm for 24 hours to get a bacterial solution. Then, take and inoculate the bacterial solution with OD₆₀₀ of 10, therein into 100 mL of M3 fermentation medium containing 33% of fish scale powder, and ferment at 37° C. to get a fermentation broth. Measure total concentration of peptides and amino acids in the fermentation broth before fermentation (0 hour) and after fermentation for 16, 24, 40, and 48 hrs.

A method of measuring the total concentration of amino acids used in the test is the same as the above method using ninhydrin reagent. A method of measuring total concentration of peptides and amino acids used is using o-Phthaldialdehyde (OPA), and described briefly below.

First, prepare Leucine standard solutions. Take and add 10 μL of Leucine standard solutions or test samples into wells of a 96-well plate. Then, add 100 μL of OPA solution into the standard solutions and the samples, and immediately measure the absorbance of the standard solutions and the samples at the wavelength of 340 nm (OD₃₄₀). Next, make a standard curve by absorbance of the standard solutions obtained, and calculate total concentration of peptides and amino acids in the respective samples tested according to the standard curve. The result obtained by the OPA method minus the total concentration of amino acids obtained by the ninhydrin reagent method leaves the total concentration of peptides in the sample tested.

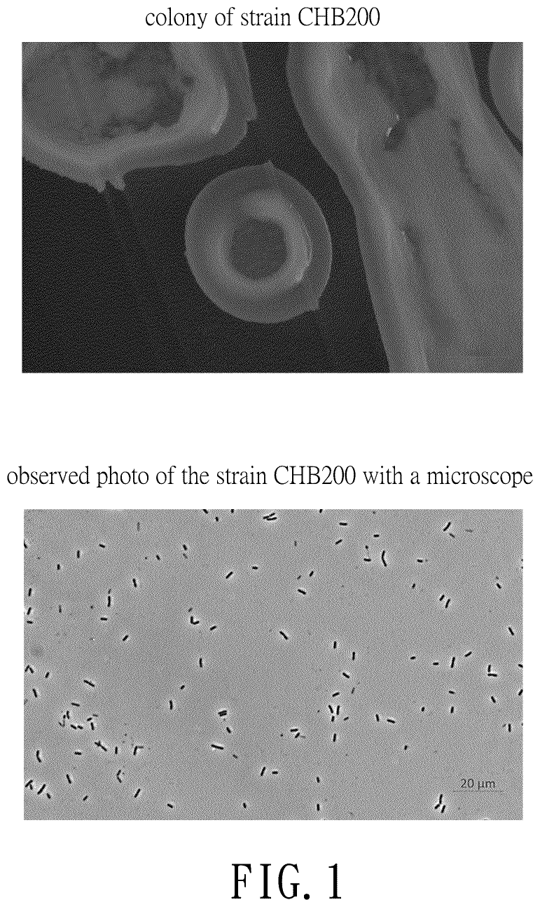

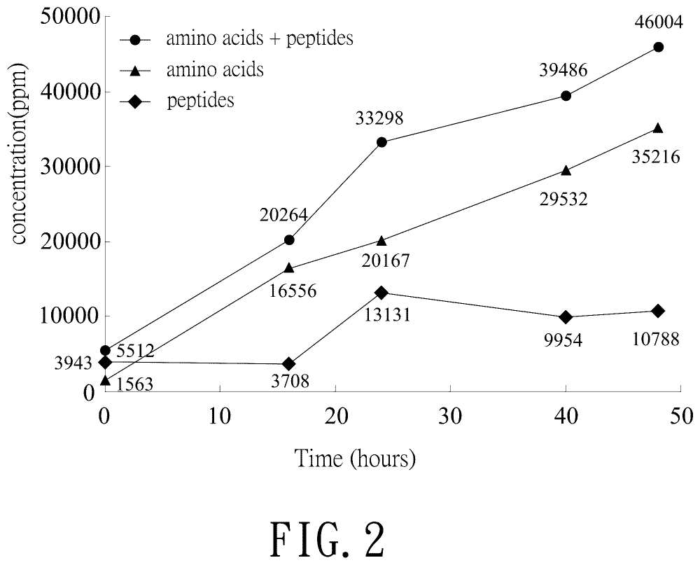

Referring to FIG. 2 and Table 2, the total concentration of amino acids and peptides in the fermentation broth is indeed increased significantly along with the increased fermentation time.

TABLE 2

| | 0 hr | 16 hrs | 24 hrs | 40 hrs | 48 hrs |
|---|---|---|---|---|---|
| Total concentration of | 5512 | 20264 | 33298 | 39486 | 46004 |
| amino acids and | | | | | |
| peptides (ppm) | | | | | |
| Total concentration of | 1569 | 16556 | 20167 | 29532 | 35216 |
| amino acids (ppm) | | | | | |
| Total concentration of | 3943 | 3708 | 13131 | 9954 | 10788 |
| peptides (ppm) | | | | | |

### Embodiment 3: Large-Scale Fermentation of Fish Scales by the Strain CHB200

First, activate the strain CHB200 to get a bacterial solution. Inoculate the bacterial solution in LB broth, adjust final volume to 100 mL, and incubate at 37° C. with shaking for 16 hours to get a saturated bacterial solution. Pour the saturated bacterial solution into a 10 L fermenter and continuously incubate at 37° C. with shaking for about 2-4 hours until OD₆₀₀ of the bacterial solution reaches 4.0. Then, pour the bacterial solution with OD₆₀₀ of 4.0 into a 100-1000 L fermenter, and incubate at 37° C. with shaking for about 2-4 hours until OD₆₀₀ reaches 4.0 to obtain a fermented bacterial solution.

Washed and cleaned fish scale materials are placed into a 100 L-10 T fermenter, and then add buffer salts and pure water to get a M3 fermentation broth containing fish scale material. The M3 fermentation broth was then sterilized at 121° C. and 1.5 atm for 30 minutes. The sterilized M3 fermentation broth is cooled down to 30-45° C., and added with the above fermented bacterial solution to get a fermentation broth. The fermentation broth is fermented at 37° C., and stirred continuously for introduction of air. The fermentation time is 12-24 hours to obtain a primary product of fermentation. A composition of the above fermentation broth contains 7-10% (w/v) of fermented bacterial solution, 15-40% (w/v) of fish scale material, 0.12% (w/v) of buffer salts, and the rest percentage of pure water. The preferred ratio of the fish scale material in the fermentation broth is 20-35% (w/v), and the preferred fermentation time is 16-20 hours. The composition of the M3 fermentation broth is shown in Table 1.

The primary product of fermentation is centrifuged at 3000-5000 rpm or 9000-11000 rpm by a decanter centrifuge or tubular centrifuge. Keep supernatant to obtain a debris-free fermentation broth.

Then, the debris-free fermentation broth is centrifuge at 9000-12000 rpm or 9000-11000 rpm by a disc centrifuge or a tubular centrifuge. Remove bacteria sedimentation to get a clear fermentation broth, and measure the total concentration of amino acids, total concentration of peptides, and nitrogen content of the clear fermentation broth.

Next, the clear fermentation broth is concentrated at 45-55° C. by a vacuum low temperature evaporator for 3-6 hours to get a concentrated fermentation broth. And measure the total concentration of amino acids, total concentration of peptides, and nitrogen content of the concentrated fermentation broth.

Methods of measuring the total concentration of amino acids and peptides are as shown in the embodiment one and the embodiment two, while a method of measuring nitrogen content is described briefly below.

Mount a homogeneous test sample into a tin foil capsule and add silicon dioxide (SiO₂) as an adsorbent. The capsule is sealed into a cylinder or a cube. Then, use an elemental analyzer (Flash 2000, Thermo Fisher Scientific) to measure nitrogen and carbon contents in the test sample. Moreover, protein content in the test sample can be estimated by multiplying the nitrogen content by a factor of 6.25 since the average nitrogen content of proteins is found to be about 16 percent.

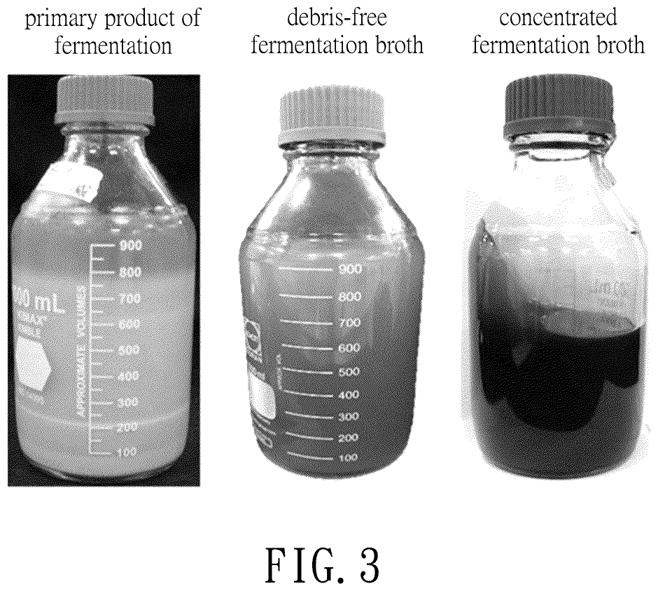

In this embodiment, a composition analysis of the clear fermentation broth is performed by SGS Certification Taiwan. The total concentration of peptides, amino acids, nitrogen content, and protein in the clear fermentation broth are respectively 28164 ppm, 73080 ppm, 1.7% and 10.56%. In the concentrated fermentation broth, the total concentration of peptides, amino acids, nitrogen content, and protein content are respectively 120510 ppm, 365015 ppm, 6.9%, and 43.125%. Refer to FIG. 3, photos of the primary product of fermentation (with debris), the debris-free fermentation broth, and the concentrated fermentation broth are provided.

Based on the above test results, it is learned that the strain CHB200 of the Bacillus velezensis, capable of decomposing fish scales, exhibits significant fish scale decomposing ability. Its efficacy in large-scale fermentation is notable due to the effective decomposition of fish scales within a short period. Thus, the strain CHB200 has potential for industrial applications. Moreover, the fermentation broth obtained from decomposition of fish scales by the strain CHB200 is rich in amino acids and peptides, and thus able to be applied to various industries such as preparation of agricultural fertilizer, raw materials for skincare products, raw materials for health supplements, even medical materials, etc. Therefore, the strain CHB200 carries substantial value for utilization.

Additional advantages and modifications will readily occur to those skilled in the art. Therefore, the invention, in its broader aspects, is not limited to the specific details and representative devices shown and described herein. Accordingly, various modifications may be made without departing from the spirit or scope of the general inventive concept as defined by the appended claims and their equivalent.

What is claimed is:

1. A mass production method using Bacillus velezensis for decomposition of fish scales comprising the steps of:
culturing a bacterial solution of Bacillus velezensis,
mixing the bacterial solution with a fish scale material to get a mixed solution, and
allowing the mixed solution to react at 37° C. for 12-24 hours for decomposition of the fish scale material; wherein the Bacillus velezensis is deposited in Leibniz Institute DSMZ-German Collection of Microorganisms and Cell Cultures GmbH with an accession number of DSM 34894.

2. The method as claimed in claim 1, wherein the mixed solution includes 7-10% (w/v) of the bacterial solution of Bacillus velezensis and 15-40% (w/v) of the fish scale material.

3. The method as claimed in claim 1, wherein time required for the step of allowing the mixed solution to react at 37° C. is 16-20 hours.

4. The method as claimed in claim 1, wherein the fish scale material includes fish scales, sodium chloride (NaCl), dipotassium phosphate (K₂HPO₄), and monopotassium phosphate (KH₂PO₄).

5. The method as claimed in claim 1, wherein the fish scale material includes fish scales, 0.5 g/L of sodium chloride (NaCl), 0.3 g/L of dipotassium phosphate (K₂HPO₄), and 0.4 g/L of monopotassium phosphate (KH₂PO₄).

6. A fish scale decomposition product obtained by the method claimed in claim 1 comprising amino acids and peptides; the fish scale decomposition product is obtained by using the Bacillus velezensis to decompose the fish scale material.

7. The product as claimed in claim 6, wherein the fish scale decomposition product is further concentrated to get a concentrated product.
